(19) United States
(12) Patent Application Publication
(10) Pub. No.: US 2013/0217897 A1
(43) Pub. Date: Aug. 22, 2013

(54) VPO CATALYST WITH LOW VANADIUM OXIDATION STATE FOR MALEIC ANDHYDRIDE PRODUCTION (D#81,745)

(71) Applicants: Zhiping Shan, The Woodlands, TX (US); Michael J. Mummey, Spring, TX (US); William S. Frazee, Spring, TX (US); Bennie A. Horrell, Spring, TX (US)

(72) Inventors: Zhiping Shan, The Woodlands, TX (US); Michael J. Mummey, Spring, TX (US); William S. Frazee, Spring, TX (US); Bennie A. Horrell, Spring, TX (US)

(73) Assignee: HUNTSMAN PETROCHEMICAL LLC, The Woodlands, TX (US)

(21) Appl. No.: 13/821,050

(22) PCT Filed: Aug. 24, 2011
(86) PCT No.: PCT/US11/48883

§ 371 Date: Mar. 20, 2013

Related U.S. Application Data
(60) Provisional application No. 61/381,747, filed on Sep. 10, 2010.

(51) Int. Cl. B01J 27/198; C07D 307/60

(52) U.S. Cl. CPC B01J 27/198; C07D 307/60
USPC 549/260; 502/209; 423/305; 549/259

(57) ABSTRACT

An oxidation catalyst comprising vanadium, phosphorus, and oxygen having average vanadium valence less than about 4.10, and a method of preparing such catalyst, is provided. The catalyst has side crush strength of at least about 5 lbs. and improved yield of maleic anhydride from n-butane between about 1% and about 6% absolute. The catalyst is formed by exposing a conventional active VPO catalyst having average vanadium valence between about 4.10 and about 4.40 to an organic solvent having a dielectric constant between about 5 and about 55 under conditions that facilitate an oxidation-reduction reaction, reducing the valence of the vanadium below 4.10.

### CROSS REFERENCE TO RELATED APPLICATIONS

This application claims priority to U.S. Provisional Application No. 61/381,747, filed Sep. 10, 2010, which is hereby incorporated by reference.

### FIELD

Embodiments described herein generally relate to a catalyst and a manufacturing process for the catalyst. More specifically, embodiments of an oxidizing catalyst and manufacturing process for the oxidizing catalyst are described.

### DESCRIPTION OF RELATED ART

Maleic anhydride is used as a raw material in the production of many products, such as synthetic resins, and may generally be prepared by catalytic oxidation of n-butane and benzene. The catalyst used for this oxidation is typically a catalyst containing vanadium, phosphorus, oxygen (VPO) and optionally a promoter component.

These catalysts are generally prepared by contacting vanadium-containing compounds with phosphorus-containing compounds and optionally promoter component containing-compounds under conditions suitable to reduce the pentavalent vanadium to the tetravalent state to thereby form a catalyst precursor containing vanadyl hydrogen phosphate and optionally the promoter component. The catalyst precursor may then be recovered and typically formed into a shaped body, such as a tablet or pellet, by compression in a die. A lubricant is ordinarily incorporated as well to aid in the tableting or pelleting process. The pellet or tablet may then be subjected to calcination to transform the catalyst precursor into an active catalyst.

Variants and different embodiments of the preparation of the active catalyst are described in the art: U.S. Pat. No. 4,567,158 discloses preparation of the catalyst precursor in the presence of an alcohol-modifying agent to form a highly porous catalyst precursor which is then converted to the active catalyst; U.S. Pat. No. 4,996,179 discloses forming the catalyst precursor into a geometric shape and calcining the shaped catalyst in an inert atmosphere at a temperature within the range of about 343° C. to 704° C. and further at an increased temperature in an oxygen containing atmosphere to produce the active catalyst; U.S. Pat. No. 5,137,860 discloses the use of three heating stages for converting the catalyst precursor into an active catalyst; U.S. Pat. No. 5,506,187 discloses preparation of the catalyst precursor in the presence of a glycol ether solvent; U.S. Pat. No. 5,530,144 discloses the use of orthophosphoric acid as the phosphorus-containing compound for producing the catalyst precursor; and U.S. Pat. No. 5,773,382 discloses the use of removable pore modification agents in the preparation of the catalyst precursor to produce a catalyst precursor exhibiting a high proportion of large pores.

Thus, there are many different methods of producing and activating these catalysts, however the main active substance in all such catalysts is believed to be vanadyl pyrophosphate (VO)₂P₂O₇. Catalytic performance of this active substance is believed to be very sensitive to preparation conditions. In the past, improvements in catalytic performance have come from processing techniques including (1) dopant addition, such as Fe, Mo, Nb, Zr as promoters, for example, as described in U.S. Pat. No. 5,158,923; and (2) catalyst structure modification including catalyst shape and pore structure inside of catalyst particles, for example, as described in U.S. Pat. No. 5,168,090. Although these and other known techniques provide catalysts having appreciable activity and selectivity in the preparation of maleic anhydride, further improvements are desirable.

### SUMMARY

Embodiments described herein provide a catalyst for oxidation of hydrocarbons, the catalyst having vanadium and phosphorus, the vanadium having an average valence state less than 4.10.

Other embodiments provide a method of making a vanadium-phosphorus catalyst, comprising contacting an active VPO catalyst having average vanadium valence of about 4.40 or less with an organic solvent having dielectric constant between about 5 and about 55, and reducing the vanadium valence of the active VPO catalyst below about 4.10 to form the vanadium-phosphorus catalyst.

Other embodiments provide a method of making a carboxylic acid anhydride, comprising disposing a catalyst comprising vanadium, phosphorus, and oxygen in a reaction vessel, the catalyst having average vanadium valence less than 4.10, contacting the catalyst with a hydrocarbon, and contacting the catalyst and the hydrocarbon with an oxygen-containing gas

### DETAILED DESCRIPTION

Surprisingly, a vanadium-phosphorus catalyst having average vanadium valence less than about 4.10 has been found to improve yield in oxidation of hydrocarbons to carboxylic acid anhydrides by 2-4 percent, absolute, or more over commercially available catalysts. The vanadium-phosphorus catalyst comprises vanadium, phosphorus, and oxygen, and optionally dopants or promoters selected from the group consisting of Zr, Mo, Nb, Cr, Fe, Zn, Ti, V, Mn, Co, Ni, and combinations thereof. The vanadium-phosphorus catalyst generally comprises vanadyl pyrophosphate (VO)₂P₂O₇ as an active ingredient, along with vanadium species in higher and lower valence states to bring the average vanadium valence below about 4.10, such as below about 4.05, for example below about 4.00. In some embodiments, the average vanadium valence is between about 3.9 and about 4.05, such as about 3.95.

References to vanadium valence herein refer to the result of an autotitration performed on a vanadium-phosphorus catalyst as described herein. A sample of vanadium-phosphorus catalyst is titrated to a millivolt endpoint with standardized potassium permanganate (KMnO₄) to oxidize all vanadium in the sample to the pentavalent V(5) state. The oxidized vanadium is then titrated to a second millivolt endpoint with standardized ferrous ammonium sulfate (Fe(NH₄)₂(SO₄)₂) to the tetravalent state V(4). The ratio of the quantity of potassium permanganate to ferrous ammonium sulfate used in the titrations is subtracted from 5 to give the vanadium valence state of the sample.

The catalyst has a ratio of phosphorus atoms to vanadium atoms between about 1.00 and about 1.15, such as between about 1.03 and 1.10, and a B.E.T. (Brunauer-Emmett-Teller) surface area of at least about 20 m²/g, such as between about 20 m²/g and about 100 m²/g, or between about 25 m²/g and about 40 m²/g, such as about 30 m²/g. Mean bulk density is typically between about 0.4 g/cc and about 1.2 g/cc, such as about 0.6 g/cc. The catalyst displays a side crush strength greater than about 5 lbs.

The catalyst may be formed into a variety of shapes to enhance reactive contact surface. The shapes may be selected from the group consisting of a cylinder, a cored cylinder, a sphere, a pellet, a trilobe, a quadrilobe, a bead, a ring, a tablet, a round trilobe, an irregular shape, or any combination thereof. The catalyst is usually shaped as part of a process for forming the catalyst that includes forming a precursor catalyst and reducing the valence state of the precursor catalyst below about 4.10, such as below about 4.05, for example below about 4.00. In some embodiment, the valence state of the precursor catalyst is reduced to between about 3.90 and about 4.05, for example about 3.95. The catalyst is usually shaped prior to reducing the valence state, but may be shaped after reducing the valence state of a powdered catalyst. A preferred round trilobe catalyst shape is described in international patent publication WO2010/047949.

The vanadium-phosphorus catalyst described above may be formed by subjecting an active vanadium phosphorus oxide (VPO) catalyst to treatment with an organic solvent. A Mars V® catalyst available from Huntsman Performance Products of The Woodlands, Tx., may be used to form the vanadium-phosphorus catalyst described above. Other similar active VPO catalysts from other manufacturers may also be used. Generally, an active VPO catalyst having average vanadium valence between about 4.10 and about 4.40, such as between about 4.15 and about 4.35, is suitable for forming into an enhanced yield catalyst as described herein.

The active VPO catalyst is contacted with an organic solvent to reduce the average valence state of the vanadium therein below about 4.10, such as below about 4.05, such as between about 3.90 and about 4.05, for example below about 4.00, such as about 3.95. The organic solvent may be a polar solvent, and may have a dielectric constant between about 5 and about 55, for example between about 6 and about 50, or between about 10 and about 50, or between about 20 and about 45, and is generally non-aqueous. In some embodiments, the solvent may be selected from the group consisting of methanol, ethanol, n-propanol, n-butanol, isopropanol, isobutanol, acetonitrile, acetone, methyl ethyl ketone (MEK), N,N-dimethylformamide (DMF), dimethyl solfoxide (DMSO), tetrafuran, ethylene glycol, propylene glycol, and any combination thereof. In one particular embodiment, ethylene glycol is used. In another embodiment, propylene glycol is used. In another embodiment, a mixture of ethylene glycol and propylene glycol is used.

The active VPO catalyst is contacted with the organic solvent for a period of time sufficient to perform an oxidation-reduction reaction. Some molecules of the solvent are oxidized while the active VPO catalyst is partially reduced. Contact time may be from about 5 minutes to about 2 days, such as from about 30 minutes to about 12 hours, for example 2 hours. Temperature for contacting is generally maintained between about room temperature and about 100° C. above the boiling point of the organic solvent, such as between about 20° C. and about 200° C., or between about 40° C. and about 140° C., for example about 80° C. Pressure is maintained between atmospheric pressure and about 5 bars, such as between atmospheric pressure and about 3 bars, for example about 2 bars. Contacting with an organic solvent transforms the active VPO catalyst into a reduced valence vanadium-phosphorus catalyst.

The reduced valence vanadium-phosphorus catalyst may be dried following contacting with the organic solvent. Drying is generally performed at a temperature and pressure, and for a time period, sufficient to remove substantially all the organic solvent. Temperature is generally between room temperature and about 400° C., for example about 350° C. Pressure is generally between atmospheric pressure and about 10 mbar (vacuum), for example about 50 mbar. Time may be between about 0.1 hours to 1 week, such as between about 0.5 hours to about 3 days, for example between about 2 hour and about 24 hours. Drying is generally performed under an atmosphere comprising air, inert gases, or a mixture thereof. Inert gases may include nitrogen, helium, argon, carbon oxides, and mixtures thereof.

Alternately, the reduced valence vanadium-phosphorus catalyst may be flushed to remove the organic solvent. In one embodiment, a fluid more easily removed by heat as compared with the organic solvent may be flowed through the wet catalyst to replace the organic solvent between catalyst particles or tablets as well as the organic solvent inside catalyst particles or tablets. In this way the reduced valence vanadium-phosphorus catalyst may be dried using conventional methods lowering drying cost and conserving energy.

Contacting the active VPO catalyst with the organic solvent and drying the reduced valence vanadium-phosphorus catalyst may be performed in the same vessel or in different vessels. Contacting with the organic solvent may be performed in a static or dynamic reactor. Exemplary static reactors include fixed bed or packed bed reactors. Exemplary dynamic reactors include fluidized bed and transport bed reactors. In one embodiment, a single reactor vessel may be used to prepare a reduced valence vanadium-phosphorus catalyst and perform an oxidation process to produce a carboxylic acid anhydride product. In another embodiment, the reduced valence vanadium-phosphorus catalyst is prepared in a first vessel and transported to a second vessel for performing an oxidation process.

In some embodiments, contacting with the organic solvent may be repeated prior to any subsequent process. For example, the active VPO catalyst may be exposed to a first organic solvent for a first period of time, after which the first organic solvent is removed, usually by flushing. The active VPO catalyst is transformed into a partially reduced vanadium-phosphorus catalyst after the first exposure. The partially reduced catalyst is then exposed to a second organic solvent for a second period of time, after which the second organic solvent is removed. The contacting cycle may be repeated any number of times, with the same or different organic solvents, to achieve the desired valence reduction. For example, in one embodiment the first organic solvent may have a low dielectric constant, for example a dielectric constant below about 20, while the second organic solvent has a high dielectric constant, for example a dielectric constant above about 40.

An active VPO catalyst meeting the above description may be subjected to the process described in U.S. Patent Publication 2010/0210858 to form a vanadium-phosphorus catalyst having average vanadium valence below about 4.10, such as below about 4.00, for example below about 3.95. Contacting the active VPO catalyst with the organic solvent removes material from the catalyst, resulting in a reduction in bulk density of between about 2% and about 20%, for example about 15%. The resulting catalyst displays improved yield of maleic anhydride from n-butane of between about 1% and about 6% absolute, for example about 2%, over the conventional active VPO catalyst.

A reduced valence vanadium-phosphorus catalyst, as described above, may be used to produce a carboxylic acid anhydride product with enhanced yield. The catalyst is disposed in a reaction vessel of any convenient type, such as a tubular or tube-shell reactor, which may have heat-exchange features, and may be constructed from glass or metal, such as carbon steel, stainless steel, iron, or nickel. The catalyst may be disposed in a static configuration, such as a fixed bed or packed bed, or a dynamic configuration, such as a fluidized bed or transport bed.

A hydrocarbon is contacted with the vanadium-phosphorus catalyst and an oxygen-containing gas to form the anhydride. The hydrocarbon generally has at least four carbon atoms, and may be linear, branched, or cyclic, and may be saturated, unsaturated, or aromatic. Maleic anhydride, for example, may be made by exposing a hydrocarbon having at least four carbon atoms in a straight chain, or a mixture of such hydrocarbons, to the vanadium-phosphorus catalyst. For maleic anhydride production, the hydrocarbon typically contains four to ten carbon atoms. Thus, butanes, pentanes, hexanes, heptanes, octanes, nonanes, and decanes, or any mixture thereof, wherein a hydrocarbon molecule has at least four carbon atoms in a straight chain, may be used. Likewise, C₄-C₁₀ alkenes and dienes. Hydrocarbons having at least four carbon atoms in a cyclic ring, for example cyclopentane, cyclopentene, benzene, or a mixture thereof, may be used. In one particular embodiment, n-butane is the hydrocarbon.

The oxygen-containing gas comprises molecular oxygen. Suitable oxygen-containing gases include, but are not limited to, air, synthetic air, molecular oxygen-enriched air, and fractionated molecular oxygen.

The reaction is typically conducted in the gas phase. The hydrocarbon is mixed with the oxygen-containing gas, and optionally with an inert gas such as nitrogen or argon, to form a gas mixture. The hydrocarbon is present in the gas mixture at a concentration between about 1 mole-percent and about 10 mole-percent. The gas mixture is contacted with the vanadium-phosphorus catalyst at a space velocity between about 100 hr⁻¹ and about 4,000 hr⁻¹, such as between about 1,000 hr⁻¹ and about 3,000 hr⁻¹, a temperature between about 300° C. and about 600° C., such as between about 325° C. and about 450° C., and pressure between atmospheric pressure and about 50 psig.

Such a process using the vanadium-phosphorus catalyst described above with n-butane as the hydrocarbon and air or oxygen gas as the oxygen-containing gas generally produces maleic anhydride at yields 1-6% absolute better than performing the same process using a commercially available active VPO catalyst.

### Examples

In a first example, several batches of commercial catalysts in round trilobe form were blended. The blend catalyst had average vanadium oxidation state (Vox) of 4.16. About 1.25 kg of this blend was loaded into a 4″ diameter glass column and total height of the catalyst bed was about 33 cm. The column skin was heated by heating element coiled around the glass column. Preheated ethylene glycol (EG, Aldrich, 99.8%) was circulated through the catalyst bed from the top using a pump and the temperature of the catalyst bed was controlled at about 100° C. EG circulation was maintained for 4 hours at a rate of about 140 ml/min.

After 4 hr circulation, the pump was stopped and EG remained in the column was drained out. Then preheated nitrogen was blown down from the top of the column to remove the remaining EG. Catalyst bed temperature was controlled by adjusting preheated nitrogen temperature and column skin temperature. Catalyst bed temperature was gradually ramped up to 300C and held there for 5 hours. After 5 hours of drying, all the heating sources were shut down and the catalyst was gradually cooled down overnight. Finally the dried catalyst was unloaded.

During unloading three catalyst samples were taken from top, middle, and bottom of the catalyst bed. All three samples were analyzed for Vox and the results were 3.83, 3.80 and 3.86, respectively, from top to bottom. The well-blended, dried catalyst had average Vox of 3.84, which is much lower than that of the original blend catalyst, 4.16.

The treated catalyst with Vox of 3.84 showed yield of maleic anhydride from n-butane of 58.3%, whereas the original catalyst having Vox of 4.16 had yield of 55.5%. This example demonstrated yield increase of about 2.8 yield point by lowering the catalyst Vox.

In a second example, several batches of commercial catalysts in round trilobe form were blended. The blend catalyst had average Vox of 4.22. About 10 lb of this blend was loaded into a 5.5″ diameter and 4′ long stainless steel column. The column was heated by heating jacket around the column. EG was circulated through the catalyst bed from the top using a pump and the temperature of the catalyst bed was ramped up to 100° C. within 1.5 hours. EG circulation was maintained for 4 hours after the catalyst bed temperature reached 100° C. at circulation rate of about 3.5 hr⁻¹ space velocity.

After 4 hr circulation, EG remaining in the column was drained out. Then preheated nitrogen was blown down from the top of the column to remove the remaining EG. Catalyst bed temperature was controlled by adjusting preheated nitrogen temperature and heating jacket temperature. Catalyst bed temperature was gradually and continuously ramped up to 350° C. and held there for 3.6 hours. After 3.6 hours of drying, all the heating sources were shut down and the catalyst was gradually cooled down overnight. Finally the dried catalyst was unloaded.

After being unloaded and blended, two catalyst samples were analyzed for Vox. Each sample of the well-blended, dried catalyst had a Vox of 3.97, which was much lower than that of the original blend catalyst, 4.22.

This EG treated catalyst was evaluated in pilot scale reactor. The reactor was 20 feet long and had inner diameter of one inch. The reactor was loaded with 6 inches of alumina at bottom, then 212 inches catalyst and about 34 inches alumina on the top. Space velocity was controlled at 1820 hr⁻¹, and n-butane feed concentration at 2.0±0.2%. Maleic anhydride yield was maintained around 59.4% at n-butane conversion of 85% after 1550 hours on-stream, which is 2.2 yield points higher than that of the original commercial catalyst.

In a third example, an original catalyst with average vanadium valence of 4.25 in trilobe tablet form was treated with a thermal bath of fresh ethylene glycol (Aldrich, 99.8%) heated to 100° C. by loading about 100 g of the original catalyst into a container with holes and immersing the container with catalyst into the hot EG bath for 2 hours. The catalyst was removed from the bath, placed into a preheated oven, and held at 100° C. for 3 hours with nitrogen purge. After 3 hours, the temperature was increased at 2° C./minute to 180° C., where It was held for 6 hours.

The valence of the resulting catalyst was measured at 4.00, and resulted in maleic anhydride yield of 57.2%, while the maleic anhydride yield of the original catalyst was 54.1%.

In a fourth example, about 40 g of a similar original catalyst was subjected to a bath of propylene glycol (Aldrich, 99.5%) at 100° C. by loading the catalyst into a similar container with holes and immersing in the PG bath for 6 hours. The catalyst was placed into a preheated oven at 100° C. for 5 hours under nitrogen purge, the temperature was increased at 2° C./minute to 170° C. and held for 3 hours, then further increased to 180° C. at 2° C./minute and held for 3 hours, then further increased to 250° C. at 2° C./minute and held for 3 hours.

The valence of the resulting catalyst was measured at 4.01, and the catalyst showed maleic anhydride yield of 60.8%, while the original catalyst showed maleic anhydride yield of 57.2%.

In a fifth example, a commercial VPO catalyst having average vanadium valence of about 4.30 was loaded into a similar container with holes and immersed in a bath of EG (Huntsman UPR grade >99.9%) at 100° C. for 4 hours. The catalyst was removed from the bath and placed in a preheated oven at 100° C. for 3 hours under nitrogen purge. After 3 hours, the temperature was increased at 2° C./minute to 180° C. and held for 3 hours, then increased at 2° C./minute to 190° C. and held for 3 hours, then increased at 2° C./minute to 250° C. and held for 3 hours.

The valence of the resulting catalyst was measured at 4.03, and the catalyst showed maleic anhydride yield of 59.7%, while the original catalyst showed maleic anhydride yield of 57.1%.

While the foregoing is directed to embodiments of the present invention, other and further embodiments of the invention may be devised without departing from the basic scope thereof, and the scope thereof is determined by the claims that follow.

1. A catalyst for oxidation of hydrocarbons, the catalyst comprising vanadium and phosphorus, wherein the vanadium has an average valence state less than about 4.10.

2. The catalyst of claim 1, wherein a ratio of phosphorus atoms to vanadium atoms is at least about 1.00.

3. The catalyst of claim 1, wherein the catalyst has a side crush strength greater than 5 pounds.

4. The catalyst of claim 1, further comprising a dopant or promoter.

5. The catalyst of claim 1, wherein the catalyst has a B.E.T. surface area of at least about 20 m³/g.

6. The catalyst of claim 3, wherein the catalyst is formed into bodies having a shape selected from the group consisting of a cylinder, a cored cylinder, a sphere, a pellet, a trilobe, a quadrilobe, a bead, a ring, a tablet, a round trilobe, an irregular shape, or a combination thereof.

7. The catalyst of claim 6, further comprising a dopant or promoter selected from the group consisting of Zr, Mo, Nb, Cr, Fe, Zn, Ti, V, Mn, Co, Ni, and combinations thereof.

8. The catalyst of claim 1, wherein the vanadium has an average valence state less than about 4.00.

9. A method of making a vanadium-phosphorus catalyst, comprising:
contacting an active VPO catalyst having average vanadium valence of about 4.40 or less with an organic solvent having dielectric constant between about 5 and about 55; and
reducing the vanadium valence of the active VPO catalyst below about 4.10 to form the vanadium-phosphorus catalyst.

10. The method of claim 9, wherein the active VPO catalyst has average vanadium valence between about 4.10 and about 4.40.

11. The method of claim 9, wherein the organic solvent is selected from the group consisting of methanol, ethanol, n-propanol, n-butanol, isopropanol, isobutanol, acetonitrile, acetone, MEK, DMF, DMSO, tetrafuran, ethylene glycol, propylene glycol, diethylene glycol, dipropylene glycol, 1,4-butanediol, glycerin, and combinations thereof.

12. The method of claim 9, further comprising repeating the contacting with organic solvent.

13. The method of claim 9, further comprising removing the organic solvent.

14. The method of claim 13, wherein removing the organic solvent comprises drying the vanadium-phosphorus catalyst.

15. The method of claim 9, wherein the vanadium valence is reduced below about 4.00.

16. The method of claim 9, wherein the active VPO catalyst is in the form of shaped bodies.

17. The method of claim 10, wherein the active VPO catalyst has an average vanadium valence between about 4.10 and about 4.35.

18. The method of claim 16, wherein the shaped bodies have a shape selected from the group consisting of cylinder, cored cylinder, sphere, trilobe, quadrilobe, bead, round trilobe, irregular shapes, and combinations thereof.

19. The method of claim 8, further comprising shaping the active VPO catalyst into bodies having a shape selected from the group consisting of cylinder, cored cylinder, sphere, trilobe, quadrilobe, bead, round trilobe, irregular shapes, and combinations thereof.

20. A method of making a carboxylic acid anhydride, comprising:
disposing a catalyst comprising vanadium, phosphorus, and oxygen in a reaction vessel, the catalyst having average vanadium valence less than 4.10;
contacting the catalyst with a hydrocarbon; and
contacting the catalyst and the hydrocarbon with an oxygen-containing gas.

21. The method of claim 20, wherein the hydrocarbon has at least four carbon atoms in a straight chain and the oxygen-containing gas comprises molecular oxygen.

22. The method of claim 21, wherein the catalyst is an active VPO catalyst with reduced average vanadium valence formed by contacting an active VPO catalyst having average vanadium valence between about 4.10 and about 4.40 with an organic solvent having a dielectric constant between about 5 and about 55.
